(19) United States
(12) Patent Application Publication
(10) Pub. No.: US 2006/0021156 A1
(43) Pub. Date: Feb. 2, 2006

(54) Composition for the oxidation dyeing of keratinous fibres containing a laccase and dyeing method using this composition

(75) Inventors: Gerard Lang, Saint Prix (FR); Jean Cotteret, Verneuil sur Seine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A.

(21) Appl. No.: 11/045,516

(22) Filed: Jan. 31, 2005

Related U.S. Application Data
(63) Continuation of application No. 09/600,136, filed on Sep. 11, 2000, now abandoned.

(30) Foreign Application Priority Data
Jan. 13, 1998 (FR) 98/00,257

(51) Int. Cl. A61K 8/00

(52) U.S. Cl. 8/405

(57) ABSTRACT

The invention relates to ready-to-use compositions for oxidation dyeing of keratin fibers, for example, human keratin fibers such as hair, comprising, in a suitable dyeing medium, at least one first oxidation base chosen from para-phenylenediamines and their acid addition salts; at least one second oxidation base chosen from para-aminophenols and their acid addition salts; at least one coupler chosen from meta-aminophanols, meta-phenylenediamines, and meta-diphenols, and their acid addition salts; and at least one enzyme of the laccase type, and to methods using these compositions.

The subject of the invention is a composition for the oxidation dyeing of keratinous fibres, and in particular of human keratinous fibres such as hair, comprising, in a medium appropriate for dyeing, at least one first oxidation base chosen from para-phenylenediamines, at least one second oxidation base chosen from para-aminophenols, at least one coupler chosen from meta-aminophenols, meta-phenylenediamines and meta-diphenols, and at least one enzyme of the laccase type, as well as the dyeing method using this composition.

It is known to dye keratinous fibres, and in particular human hair, with dyeing compositions containing precursors for oxidation dyeing, in particular ortho- and para-phenylenediamines, ortho- or para-aminophenols, heterocyclic bases generally called oxidation bases. The precursors for oxidation dyeing, (oxidation bases), are colourless or weakly coloured compounds which, combined with oxidizing products, can give rise to dye and coloured compounds by a process of oxidative condensation.

It is also known that the shades obtained with these oxidation bases can be varied by combining them with couplers or colour modifiers, the latter being chosen in particular from aromatic meta-diamines, meta-aminophenols, meta-diphenols and certain heterocyclic compounds.

The variety of molecules used in oxidation bases and couplers allows a rich palette of colours to be obtained.

The so-called “permanent” colour obtained by means of these oxidation dyes should moreover satisfy a number of requirements. Thus, it should have no drawbacks from the toxicological point of view, it should make it possible to obtain shades of the desired intensity and it should exhibit good resistance towards external agents (light, adverse weather conditions, washing, permanent-waving, perspiration, rubbing).

The dyes should also make it possible to cover grey hair, and thus should be the least selective possible, that is to say they should make it possible to obtain the smallest possible differences in colour all along the same keratinous fibre, which may indeed be differently sensitized (i.e. damaged) between its tip and its root.

The oxidation dyeing of keratinous fibres is generally carried out in an alkaline medium, in the presence of hydrogen peroxide. However, the use of alkaline media in the presence of hydrogen peroxide has the disadvantage of causing substantial degradation of the fibres, as well as decolouring of the keratinous fibres which is not always desirable.

The oxidation dyeing of keratinous fibres can also be carried out with the aid of oxidizing systems different from hydrogen peroxide such as enzymatic systems. Thus, it has already been proposed in U.S. Pat. No. 3,251,742, and Patent Applications FR-A-2,112,549, FR-A-2,694,018, EP-A-0,504,005, WO95/07988, WO95/33836, WO95/33837, WO96/00290, WO97/19998 and WO97/19999 to dye keratinous fibres with compositions comprising at least one oxidation dye, or at least one melanin precursor, in combination with enzymes of the laccase type, the said compositions being brought into contact with atmospheric oxygen. These dyeing formulations, although used under conditions which do not cause degradation of the keratinous fibres comparable to that caused by dyeings carried out in the presence of hydrogen peroxide, lead to colours which are still inadequate both from the point of view of homogeneity of the colour distributed along the fibre (unison), from the point of view of chromaticity (luminosity) and of the dyeing power.

Now, the applicant has now just discovered that it is possible to obtain novel dyes which are capable of giving intense and chromatic colours, without causing significant degradation of the keratinous fibres, which are not very selective and which are quite resistant to various attacks to which the fibres may be subjected, by combining at least one first oxidation base chosen from para-phenylene-diamines, at least one second oxidation base chosen from para-aminophenols, at least one coupler chosen from meta-aminophenols, meta-phenylenediamines and meta-diphenols, and at least one enzyme of the laccase type.

This discovery forms the basis of the present invention.

The first subject of the invention is therefore a ready-to-use composition for the oxidation dyeing of keratinous fibres, and in particular human keratinous fibres such as hair, characterized in that it comprises, in a medium appropriate for dyeing:
at least one first oxidation base chosen from para-phenylenediamines,
at least one second oxidation base chosen from para-aminophenols,
at least one coupler chosen from meta-aminophenols, meta-phenylenediamines and meta-diphenols, and
at least one enzyme of the laccase type;
   it being understood that:

when the said composition contains para-phenylenediamine or one of its addition salts with an acid as first oxidation base and para-aminophenol as second oxidation base, then the coupler cannot be chosen from 4-chlororesorcinol, meta-phenylenediamine, and its addition salts with an acid; and

when the said composition contains para-tolylene-diamine as first oxidation base and para-aminophenol as second oxidation base, then the coupler cannot be chosen from 5-amino-2-methylphenol and resorcinol.

The ready-to-use dyeing composition in accordance with the invention gives intense colours which exhibit low selectivity and excellent properties of resistance both to atmospheric agents such as light and adverse weather conditions and to perspiration and various treatments to which the hair may be subjected (washing, permanent deformation).

The subject of the invention is also a method for the oxidation dyeing of keratinous fibres using this ready-to-use dyeing composition.

The laccase(s) used in the ready-to-use dye composition in accordance with the invention may be chosen in particular from laccases of plant origin, animal origin, fungal origin (yeasts, moulds, fungi) or bacterial origin, organisms which may be of mono- or pluricellular origin. The laccase(s) used in the ready-to-use dyeing composition in accordance with the invention can also be obtained by biotechnology.

Among the laccases of plant origin which can be used according to the invention, there may be mentioned the laccases produced by plants which perform chlorophyll synthesis such as those indicated in Patent Application FR-A-2,694,018.

In particular there may be mentioned the laccases present in the extracts of Anacardiaceae such as for example the extracts of Magnifera indica, Schinus molle or Pleiogynium timoriense, in the extracts of Podocarpaceae, Rosmarinus off., Solanum tuberosum, Iris sp., Coffea sp., Daucus carrota, Vinca minor, Persea americana, Catharenthus roseus, Musa sp., Malus pumila, Gingko biloba, Monotropa hypopithys (Indian pipe), Aesculus sp., Acer pseudoplatanus, Prunus persica and Pistacia palaestina.

Among the laccases of fungal origin, optionally obtained by biotechnology, which can be used according to the invention, there may be mentioned the laccase(s) derived from Polyporus versicolor, Rhizoctonia practicola and Rhus vernicifera as described for examples in Patent Applications FR-A-2,112,549 and EP-A-504005, the laccases described in Patent Application WO95/07988, WO95/33836, WO95/33837, WO96/00290, WO97/19998 and WO97/19999, whose content is an integral part of the present description, such as for example the laccase(s) derived from Scytalidium, Polyporus pinsitus, Myceliophtora thermophila, Rhizoctonia solani, Pyricularia orizae, and variants thereof. There may also be mentioned the laccase(s) derived from Tramates versicolor, Fomes fomentarius, Chaetomium thermophile, Neurospora crassa, Colorius versicol, Botrytis cinerea, Rigidoporus lignosus, Phellinus noxius, Pleurotus ostreatus, Aspergillus nidulans, Podospora anserina, Agaricus bisporus, Ganoderma lucidum, Glomerella cingulata, Lactarius piperatus, Russula delica, Heterobasidion annosum, Thelephora terrestris, Cladosporium cladosporiodes, Cerrena unicolor, Coriolus hirsutus, Ceriporiopsis subvermispora, Coprinus cinereus, Panaeolus papilionaceus, Panaeolus sphinctrinus, Schizophyllum commune, Dichomitius squalens, and variants thereof.

The laccases of fungal origin optionally obtained by biotechnology will be preferably chosen.

The enzymatic activity of the laccases used in accordance with the invention and which have syringaldazine among their substrate can be defined from the oxidation of syringaldazine under aerobic conditions. The Lacu unit corresponds to the quantity of enzyme catalysing the conversion of 1 mmol of syringaldazine per minute at pH 5.5 and at a temperature of 30° C. The unit U corresponds to the quantity of enzyme producing a delta absorbance of 0.001 per minute, at a wavelength of 530 nm, using syringaldazine as substrate, at 30° C. and at a pH of 6.5. The enzymatic activity of the laccases of the invention can also be defined from the oxidation of para-phenylenediamine. The lacu unit corresponds to the quantity of enzyme producing a delta absorbance of 0.001 per minute, at a wavelength of 496.5 nm, using para-phenylenediamine as substrate (64 mM), at 30° C. and at a pH of 5.

According to the invention, it is preferable to determine the enzymatic activity in lacu units.

The quantity of laccase(s) present in the ready-to-use dyeing composition in accordance with the invention will vary according to the nature of the laccase(s) used. Preferably, the quantity of laccase(s) is between 0.5 and 200 Lacu approximately (that is to say between 10,000 and 4×10⁶ U units approximately or that is to say between 20 and 2×10⁶ lacu units) per 100 g of dyeing composition.

Among the para-phenylenediamines which can be used as first oxidation base in the ready-to-use dyeing composition in accordance with the invention, there may be mentioned in particular the compounds of the following formula (I), and their addition salts with an acid: 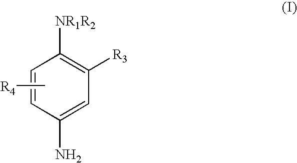
in which:
R₁ represents a hydrogen atom, a C₁-C₄ alkyl radical, a monohydroxy(C₁-C₄ alkyl) radical, a polyhydroxy-(C₂-C₄ alkyl) radical, a (C₁-C₄)alkoxy(C₁-C₄)alkyl radical, a C₁-C₄ alkyl radical substituted with a nitrogen-containing group, a phenyl radical or a 4′-aminophenyl radical;
R₂ represents a hydrogen atom, a C₁-C₄ alkyl radical, a monohydroxy(C₁-C₄ alkyl) radical, a polyhydroxy(C₂-C₄ alkyl) radical, a (C₁-C₄)alkoxy(C₁-C₄)alkyl radical or a C₁-C₄ alkyl radical substituted with a nitrogen-containing group;
R₃ represents a hydrogen atom, a halogen atom such as a chlorine, bromine, iodine or fluorine atom, a C₁-C₄ alkyl radical, a monohydroxy(C₁-C₄ alkyl) radical, a hydroxy(C₁-C₄ alkoxy) radical, an acetylamino(C₁-C₄ alkoxy) radical, a mesylamino(C₁-C₄ alkoxy) radical or a carbamoylamino(C₁-C₄ alkoxy) radical,
R₄ represents a hydrogen or halogen atom or a C₁-C₄ alkyl radical.

Among the nitrogen-containing groups of formula (I) above, there may be mentioned in particular the amino, mono(C₁-C₄)alkylamino, (C₁-C₄)dialkylamino, (C₁-C₄)trialkylamino, monohydroxy(C₁-C₄)alkylamino, imidazolinium and ammonium radicals.

Among the para-phenylenediamines of formula (I) above, there may be mentioned more particularly para-phenylenediamine, para-tolylenediamine, 2-chloro-para-phenylenediamine, 2,3-dimethyl-para-phenylene-diamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylene-diamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, and their addition salts with an acid.

Among the para-phenylenediamines of formula (I) above, there are most particularly preferred para-phenylenediamine, para-tolylenediamine, 2-isopropyl-para-phenylenediamine, 2-β-hydroxyethyl-para-phenylenediamine, 2-β-hydroxyethyloxy-para-phenylene-diamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine, and their addition salts with an acid.

The para-phenylenediamine(s) used as first oxidation base preferably represent from 0.0005 to 12% by weight approximately of the total weight of the ready-to-use dyeing composition in accordance with the invention, and still more preferably from 0.005 to 6% by weight approximately of this weight.

Among the para-aminophenols which can be used as second oxidation base in the dyeing compositions in accordance with the invention, there may be mentioned in particular the compounds corresponding to the following formula (II), and their addition salts with an acid: 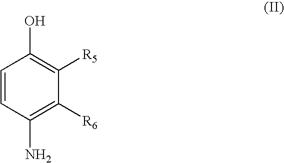
in which:
R₅ represents a hydrogen or halogen atom, a C₁-C₄ alkyl, C₁-C₄ monohydroxyalkyl, (C₁-C₄) alkoxy(C₁-C₄)alkyl, C₁-C₄ aminoalkyl or hydroxy(C₁-C₄)alkylamino-(C₁-C₄)alkyl radical,
R₆ represents a hydrogen or halogen atom, a C₁-C₄ alkyl, C₁-C₄ monohydroxyalkyl, C₂-C₄ polyhydroxyalkyl, C₁-C₄ aminoalkyl, C₁-C₄ cyanoalkyl or (C₁-C₄)alkoxy(C₁-C₄)alkyl radical,
   it being understood that at least one of the R₅ or R₆ radicals represents a hydrogen atom.

Among the para-aminophenols of formula (II) above, there may be mentioned more particularly para-aminophenol, 4-amino-3-methylphenol, 4-amino-3-fluoro-phenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and their addition salts with an acid.

The para-aminophenol(s) used as second oxidation base preferably represent from 0.0005 to 12% by weight approximately of the total weight of the ready-to-use dyeing composition in accordance with the invention, and still more preferably from 0.005 to 6% by weight approximately of this weight.

The meta-aminophenol(s) which may be used as coupler in the ready-to-use dyeing composition in accordance with the invention are preferably chosen from the compounds of the following formula (III), and their addition salts with an acid: 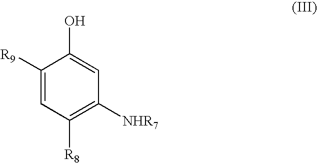
in which:
R₇ represents a hydrogen atom, a C₁-C₄ alkyl, C₁-C₄ monohydroxyalkyl or C₂-C₄ polyhydroxyalkyl radical,
R₈ represents a hydrogen atom, a C₁-C₄ alkyl or C₁-C₄ alkoxy radical or a halogen atom chosen from chlorine, bromine or fluorine,
R₉ represents a hydrogen atom, a C₁-C₄ alkyl, C₁-C₄ alkoxy, C₁-C₄ monohydroxyalkyl, C₂-C₄ polyhydroxy-alkyl, C₁-C₄ monohydroxyalkoxy or C₂-C₄ polyhydroxy-alkoxy radical.

Among the meta-aminophenols of formula (III) above, there may be mentioned more particularly meta-aminophenol, 5-amino-2-methoxyphenol, 5-amino-2-(β-hydroxyethyloxy)phenol, 5-amino-2-methylphenol, 5-N-(β-hydroxyethyl)amino-2-methylphenol, 5-N-(β-hydroxy-ethyl)amino-4-methoxy-2-methylphenol, 5-amino-4-methoxy-2-methylphenol, 5-amino-4-chloro-2-methylphenol, 5-amino-2,4-dimethoxyphenol, 5-(γ-hydroxy-propylamino)-2-methylphenol, and their addition salts with an acid.

The meta-phenylenediamine(s) which can be used as coupler in the ready-to-use dyeing composition in accordance with the invention are preferably chosen from the compounds of the following formula (IV), and their addition salts with an acid: 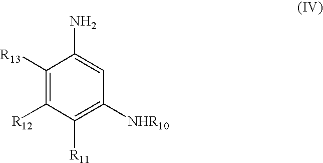
in which:
R₁₀ represents a hydrogen atom, a C₁-C₄ alkyl, C₁-C₄ monohydroxyalkyl or C₂-C₄ polyhydroxyalkyl radical;
R₁₁ and R₁₂, which are identical or different, represent a hydrogen atom, a C₁-C₄ alkyl, C₁-C₄ monohydroxyalkoxy or C₂-C₄ polyhydroxyalkoxy radical;
R₁₃ represents a hydrogen atom, a C₁-C₄ alkoxy, C₁-C₄ aminoalkoxy, C₁-C₄ monohydroxyalkoxy or C₂-C₄ polyhydroxyalkoxy radical or a 2,4-diaminophenoxy-alkoxy radical.

Among the meta-phenylenediamines of formula (IV) above, there may be mentioned more particularly 2,4-diaminobenzene, 3,5-diamino-1-ethyl-2-methoxy-benzene, 3,5-diamino-2-methoxy-1-methylbenzene, 2,4-diamino-1-ethoxybenzene, 1,3-bis(2,4-diamino-phenoxy)propane, bis(2,4-diaminophenoxy)methane, 1-(β-aminoethyloxy)-2,4-diaminobenzene, 2-amino-1-(β-hydroxyethyloxy)-4-methylaminobenzene, 2,4-diamino-1-ethoxy-5-methylbenzene, 2,4-diamino-5-(β-hydroxyethyloxy)-1-methylbenzene, 2,4-diamino-1-(β,γ-dihydroxypropyloxy)benzene, 2,4-diamino-1-(β-hydroxy-ethyloxy)benzene, 2-amino-4-N-(β-hydroxyethyl)amino-1-methoxybenzene, and their addition salts with an acid.

The meta-diphenol(s) which can be used as coupler in the ready-to-use dyeing composition in accordance with the invention are preferably chosen from the compounds of the following formula (V), and their addition salts with an acid: 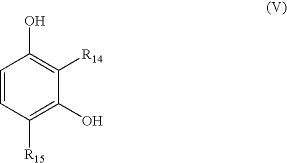
in which:
R₁₄ and R₁₅, which are identical or different, represent a hydrogen atom, a C₁-C₄ alkyl radical or a halogen atom chosen from chlorine, bromine or fluorine.

Among the meta-diphenols of formula (V) above, there may be mentioned more particularly 1,3-dihydroxybenzene, 2-methyl-1,3-dihydroxybenzene, 4-chloro-1,3-dihydroxybenzene, 2-chloro-1,3-dihydroxy-benzene, and their addition salts with an acid.

The coupler(s) used in accordance with the invention, that is to say the meta-phenylenediamine(s) and/or the meta-aminophenol(s) and/or the meta-diphenol(s) preferably represent from 0.0001 to 10% by weight approximately of the total weight of the ready-to-use dyeing composition, and still more preferably from 0.005 to 5% by weight approximately of this weight.

The ready-to-use dyeing composition in accordance with the invention may, in addition, contain one or more additional couplers different from the couplers mentioned above and/or one or more direct dyes, in particular in order to modify the shades or to increase their shimmer.

In general, the addition salts with an acid which can be used in the context of the dyeing compositions of the invention (oxidation bases and couplers) are in particular chosen from hydrochlorides, hydrobromides, sulphates and tartrates, lactates and acetates.

The medium appropriate for dyeing (or carrier) of the ready-to-use dyeing composition in accordance with the invention generally consists of water or of a mixture of water and of at least one organic solvent in order to solubilize the compounds which might not be sufficiently soluble in water.

The pH of the ready-to-use composition in accordance with the invention is chosen such that the enzymatic activity of the laccase is sufficient. It is generally between 4 and 11 approximately, and preferably between 6 and 9 approximately. It may be adjusted to the desired value using acidifying or alkalinizing agents normally used in dyeing keratinous fibres.

The ready-to-use dyeing composition in accordance with the invention may also contain various adjuvants conventionally used in hair dyeing compositions, such as anionic, cationic, nonionic, amphoteric or zwitterionic surfactants or mixtures thereof, polymers, antioxidants, enzymes different from the laccases used in accordance with the invention, such as for example peroxidases or oxidoreductases containing 2 electrons, penetrating agents, sequestering agents, perfumes, buffers, dispersing agents, thickening agents, film-forming agents, preservatives, opacifying agents or vitamins.

Of course, persons skilled in the art will be careful to choose this or these optional additional compounds such that the advantageous properties intrinsically attached to the ready-to-use dyeing composition in accordance with the invention are not, or substantially not, impaired by the addition(s) envisaged.

The ready-to-use dyeing composition in accordance with the invention can be provided in various forms, such as in the form of liquids, creams, gels, optionally pressurized, or in any other form appropriate for dyeing keratinous fibres, in particular human hair. In this case, the oxidation dyes and the enzyme(s) of laccase type are present in the same ready-to-use composition, and consequently the said composition should be free of gaseous oxygen, so as to avoid any premature oxidation of the oxidation dyes.

The subject of the invention is also a method of dyeing keratinous fibres, and in particular human keratinous fibres such as hair, using the ready-to-use dyeing composition as defined above.

According to this method, at least one ready-to-use dyeing composition as defined above is applied to the fibres for a sufficient time to develop the desired colour, after which they are rinsed, optionally washed with shampoo, rinsed again and dried.

The time necessary for the development of the colour on the keratinous fibres is generally between 3 and 60 minutes and still more precisely 5 and 40 minutes.

According to one particular embodiment of the invention, the method comprises a preliminary step consisting in storing in a separate form, on the one hand, a composition (A) comprising, in a medium appropriate for dyeing, at least one first oxidation base chosen from para-phenylenediamines, at least one second oxidation base chosen from para-aminophenols, at least one coupler chosen from meta-aminophenols, meta-phenylenediamines and meta-diphenols, and on the other hand, a composition (B) containing, in a medium appropriate for dyeing, at least one enzyme of the laccase type, and then in mixing them at the time of use before applying this mixture to the keratinous fibres.

Another subject of the invention is a multi-compartment device or dyeing (kit) or any other multi-compartment packaging system in which a first comparment contains the composition (A) as defined above and a second compartment contains a composition (B) as defined above. These devices may be equipped with a means which makes it possible to deliver the desired mixture to the hair, such as the devices described in Patent FR-2,586,913 in the name of the applicant.

The following examples are intended to illustrate the invention without limiting the scope as a result.

### EXAMPLES

### Dyeing Examples 1 o 3

The following ready-to-use dyeing compositions were prepared (contents in grams):

| | COMPOSITION | | |
|---|---|---|---|
| | 1 | 2 | 3 |
| para-Phenylenediamine | 0.17 | 0.17 | 0.17 |
| (first oxidation base) | | | |
| para-Aminophenol | 0.08 | 0.08 | 0.08 |
| (second oxidation base) | | | |
| 2-methyl-5-aminophenol | 0.14 | — | — |
| (coupler) | | | |
| 2,4-diamino-1-(β-hydroxy- | — | 0.14 | |
| ethyloxy)benzene | | | |
| dihydrochloride (coupler) | | | |
| 1,3-dihydroxybenzene | — | — | 0.12 |
| (coupler) | | | |
| Laccase derived from | 1.8 | 1.8 | 1.8 |
| Rhus vernicifera | | | |
| containing 180 units/mg | | | |
| sold by the company Sigma | | | |
| Common dye carrier (*) | (*) | (*) | (*) |
| Demineralized water qs | 100 g | 100 g | 100 g |

(*): Common dye carrier:

(C₈-C₁₀)Alkyl polyglucoside in aqueous 8.0 g solution containing 60% of active

substance (AS) sold under the name ORAMIX CGL110® by the company SEPPIC

| | |
|---|---|
| Ethanol | 20.0 g |
| pH agent qs | pH = 6.5 |

Each of the ready-to-use dyeing compositions described above was applied to locks of natural grey hair which is 90% white for 40 minutes, and at a temperature of 30° C. The hair was then rinsed, and then dried.

The hair was dyed in the shades presented in the table below:

| EXAMPLE | Shade obtained |
|---|---|
| 1 | Mahogany blonde |
| 2 | Dark purple blonde |
| 3 | Golden blonde |

In the dyeing compositions described above, Rhus vernicifera laccase at 180 units/mg, sold by the company Sigma, can be replaced by 1.0 g of Pyricularia orizae laccase at 100 units/mg sold by the company ICN.

1-28. (canceled)

29. A composition for the oxidation dyeing of keratinous fibers, comprising:
(a) at least one first oxidation base chosen from para-phenylenediamines and their acid addition salts;
(b) at least one second oxidation base chosen from para-aminophenols and their acid addition salts;
(c) at least one coupler chosen from meta-aminophanols, meta-phenylenediamines, and meta-diphenols, and their acid addition salts; and
(d) at least one enzyme of the laccase type;
provided that when said composition comprises at least one second oxidation base chosen from para-aminophenols, then the coupler is not chosen from 4-chlororesorcinol, meta-phenylenediamine, and their acid addition salts; and
provided that when said composition comprises at least one first oxidation base chosen from para-tolylene-diamines and at least one second oxidation base chosen from para-aminophenols, then the coupler is not chosen from 5-amino-2-methylphenol and resorcinol.

30. The composition according to claim 29, wherein said keratinous fibers are human hair.

31. The composition according to claim 29, wherein said at least one enzyme of the laccase type is chosen from laccases of plant origin, animal origin, fungal origin and bacterial origin, and laccases obtained by biotechnology.

32. The composition according to claim 29, wherein said at least one enzyme of the laccase type is of plant origin and is chosen from the laccases extracted from plants chosen from Anacardiaceae, Podocarpaceae, Rosmarinus off., Solanum tuberosum, Iris sp., Coffea sp., Daucus carrota, Vinca minor, Persea americana, Catharenthus roseus, Musa sp., Malus pumila, Gingko biloba, Monotropa hypopithys, Aesculus sp., Acer pseudoplatanus, Prunus persica, and Pistacia palaestina.

33. The composition according to claim 29, wherein said at least one enzyme of the laccase type is of microbial origin or obtained by biotechnology.

34. The composition according to claim 29, wherein said at least one enzyme of the laccase type is chosen from the laccases derived from fungi chosen from Polyporus versicolor, Rhizoctonja praticola, Rhus vernicifera, Scytalidium, Polyporus pinsitus, Myceliophtora thermophila, Rhizoctonia solani, Pyricularia orizae, Tramates versicolor, Fomes fomentarius, Chaetomium thermophile, Neurospora crassa, Colorius versicol, Botrytis cinerea, Rigidoporus lignosus, Phellinus noxius, Pleurotus ostreatus, Aspergillus nidulans, Podospora anserina, Agaricus bisporus, Ganoderma lucidum, Glomerella cingulata, Lactarius piperatus, Russula delica, Heterobasidion annosum, Thelephora terrestris, Cladosporium cladosporioides, Cerrena unicolor, Coriolus hirsutus, Ceriporiopsis subvermispora, Coprinus cinereus, Panaeolus papilionaceus, Panaeolus sphinctrinus, Schizophyllum commune, Dichomitius squalens, and variants of all said fungi.

35. The composition according to claim 29, wherein said at least one enzyme of the laccase type is present in a quantity ranging from 0.5 to 200 Lacu units per 100 g of said composition.

36. The composition according to claim 29, wherein said para-phenylene-diamines are chosen from the compounds of the following formula (I) and their acid addition salts: 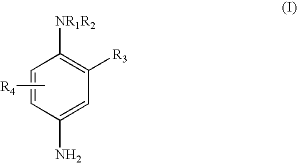
in which:
R₁ is chosen from hydrogen, C₁-C₄ alkyl groups, mono hydroxy(C₁ C₄ alkyl) groups, polyhydroxy-(C₂-C₄ alkyl) groups, (C₁-C₄)alkoxy(C₁-C₄)alkyl groups, C₁-C₄ alkyl groups substituted with a nitrogen-containing groups, phenyl groups, and 4′-aminophenyl groups;
R₂ is chosen from hydrogen, C₁-C₄ alkyl groups, monohydroxy(C₁-C₄ alkyl) groups, polyhydroxy(C₂-C₄ alkyl) groups, (C₁-C₄)alkoxy(C₁-C₄)alkyl groups, and C₁-C₄ alkyl groups substituted with a nitrogen-containing group;
R₃ is chosen from hydrogen, halogens, C₁-C₄ alkyl groups, monohydroxy (C₁-C₄ alkyl) groups, hydroxy(C₁-C₄ alkoxy) groups, acetylamino(C₁-C₄ alkoxy) groups, mesylamino (C₁-C₄ alkoxy) groups, and carbamoylamino(C₁-C₄ alkoxy) groups;
R₄ is chosen from hydrogen, halogens, and C₁-C₄ alkyl groups.

37. The composition according to claim 36, wherein said para-phenylene-diamines of formula (I) are chosen from para-phenylenediamine, para-tolylenediamine, 2-chloro-para-phenylenediamine, 2,3-dimethyl-para-phenylene-diamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl) amino-2-methyl-aniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylene-diamine, N,N-dimethyl-3-methyl-para-phenylenediamine, N,N-(ethyl-β-hydroxyethyl)-para-phenylenediamine, N-(β,γ-dihydroxy propyl)-para-phenylenediamine, N-(4′-aminophenyl)-para-phenylenediamine, N-phenyl-paraphenylenediamine, 2-β-hydroxyethyl oxypara-phenylenediamine, 2-β-acetylaminoethyloxy-para-phenylenediamine, N-(β-methoxyethyl)-para-phenylenediamine, and their acid addition salts.

38. The composition according to claim 29, wherein said first oxidation base is present in a concentration ranging from about 0.0005% to about 12% by weight of the total weight of said composition.

39. The composition according to claim 29, wherein said para-aminophenols are chosen from the compounds of formula (II), and their acid addition salts: 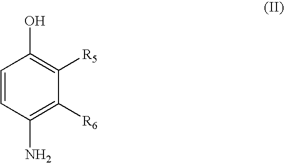
in which:
R₅ is chosen from hydrogen, halogens, C₁-C₄ alkyl groups, monohydroxy(C₁-C₄ alkyl) groups, (C₁-C₄)alkoxy(C₁-C₄-alkyl groups, amino(C₁-C₄ alkyl) groups, and hydroxy (C₁-C₄)alkylamino-(C₁-C₄ alkyl) groups;
R₆ is chosen from hydrogen, halogens, C₁-C₄ alkyl groups, monohydroxy (C₁-C₄ alkyl) groups, polyhydroxy (C₂-C₄ alkyl) groups, amino(C₁-C₄ alkyl) groups, cyano(C₁-C₄ alkyl) groups, and (C₁-C₄)alkoxy(C₁-C₄)alkyl groups;
wherein at least one of R₅ or R₆ is a hydrogen atom.

40. The composition according to claim 39, wherein said para-aminophenols of formula (II) are chosen from para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxy methylphenol, 4-amino-2-aminomethylphenol, 4-amino-2-(β-hydroxy-ethylaminomethyl)phenol, 4-amino-2-fluoro-phenol, and their acid addition salts.

41. The composition according to claim 29, wherein said second oxidation base is present in a concentration ranging from about 0.0005% to about 12% by weight of the total weight of said composition.

42. The composition according to claim 29, wherein said meta-amino-phenols are chosen from the compounds of the following formula (III), and their acid addition salts: 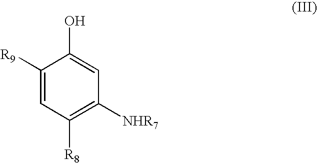
in which:
R₇ is chosen from hydrogen, C₁-C₄ alkyl groups, C₁-C₄ monohydroxyalkyl groups, and C₂-C₄ polyhydroxyalkyl groups;
R₈ is chosen from hydrogen, C₁-C₄ alkyl groups, C₁-C₄ alkoxy groups, and halogens chosen from chlorine, bromine, and fluorine;
R₉ is chosen from hydrogen, C₁-C₄ alkyl groups, C₁-C₄ alkoxy groups, C₁-C₄ monohydroxyalkyl groups, C₂-C₄ polyhydroxyalkyl groups, C₁-C₄ monohydroxyalkoxy groups, and C₂-C₄ polyhydroxy-alkoxy groups.

43. The composition according to claim 42, wherein said meta-aminophenols of formula (III) are chosen from meta-aminophenol, 5-amino-2-methoxyphenol, 5-amino-2-(β hydroxyethyloxy)phenol, 5-amino-2-methylphenol, 5-N-β hydroxyethyl)amino-2-methylphenol, 5-N-(β-hydroxyethyl)amino-4-methoxy-2-methylpheno1, 5-amino-4-methoxy-2-methylphenol, 5-amino-4-chloro-2-methylphenol, 5-amino-2,4-dimethoxyphenol, 5-(β-hydroxy-propylamino)-2-methylphenol, and their acid addition salts.

44. The composition according to claim 29, wherein said meta-phenylene-diamines are chosen from the compounds of the following formula (IV), and their acid addition salts: 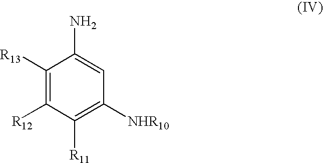
in which:
R₁₀ is chosen from hydrogen, C₁-C₄ alkyl groups, C₁-C₄ monohydroxyalkyl groups, and C₂-C₄ polyhydroxyalkyl groups;
R₁₁ and R₁₂, which may be identical or different, are each chosen from hydrogen, C₁-C₄ alkyl groups, C₁-C₄ monohydroxyalkoxy groups, and C₂-C₄ polyhydroxyalkoxy groups,
R₁₃ is chosen from hydrogen, C₁-C₂ alkoxyl groups, C₁-C₄ aminoalkoxy groups, C₁-C₄ monohydroxyalkoxy groups, C₂-C₄ polyhydroxy-alkoxy groups, and 2,4-diaminophenoxy-alkoxy groups.

45. The composition according to claim 44, wherein said meta-phenylene-diamines of formula (IV) are chosen from 2,4-diaminobenzene, 3,5-diamino-1-ethyl-2-methoxybenzene, 3,5-diamino-2-methoxy-1-methylbenzene, 2,4-diamino-1-ethoxybenzene, 1,3-bis(2,4-diaminophenoxy)propane, bis(2,4-diaminophenoxy)methane, 1-(β-aminoethyloxy)-2,4-diaminobenzene, 2-amino-1-(β-hydroxyethyloxy)-4-methyl-aminobenzene, 2,4-diamino-1-ethoxy-5-methyl-benzene,2,4-diamino-5-(β-hydroxyethyloxy)-1-methyl-benzene, 2,4-diamino-1-(β,γ-dihydroxypropyloxy) benzene, 2,4-diamino-1-(β-hydroxy-ethyloxy)benzene, 2-amino-4-N-(β-hydroxyethyl)amino-1-methoxybenzene, and their acid addition salts.

46. The composition according to claim 29, wherein said meta-diphenols are chosen from the compounds of the following formula (V), and their acid addition salts: 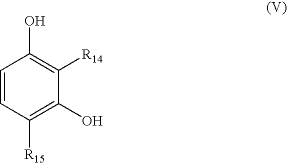
in which:
R₁₄ and R₁₅, which may be identical or different, are each chosen from hydrogen, C₁-C₄ alkyl groups, and halogens chosen from chlorine, bromine, and fluorine.

47. The composition according to claim 46, wherein said meta-diphenols of formula (V) are chosen from 1,3-dihydroxybenzene, 2-methyl-1,3-dihydroxybenzene, 4-chloro-1,3-dihydroxybenzene, 2-chloro-1,3-dihydroxybenzene, and their acid addition salts.

48. The composition according to claim 29, wherein said at least one coupler is present in a concentration ranging from about 0.0001% to about 10% by weight of the total weight of said composition.

49. The composition according to claim 29, wherein said acid addition salts of said at least one first and second oxidation bases are chosen from hydrochlorides, hydrobromides, sulphates, tartrates, lactates, and acetates.

50. The composition according to claim 29, wherein said acid addition salts of said at least one coupler are chosen from hydrochlorides, hydrobromides, sulphates, tartrates, lactates, and acetates.

51. The composition according to claim 29, further comprising at least one carrier appropriate for dyeing keratinous fibers.

52. The composition according to claim 51, wherein said at least one carrier is chosen from water and at least one organic solvent.

53. The composition according to claim 29, wherein said composition has a pH ranging from about 4 to about 11.

54. A method of dyeing keratinous fibers, comprising the step of applying at least one dyeing composition to said keratinous fibers for a sufficient time to achieve a desired coloration, wherein said at least one dyeing composition comprises:
(a) at least one first oxidation base chosen from para-phenylenediamines and acid addition salts of said para-phenyidiamines;
(b) at least one second oxidation base chosen from para-aminophenols and acid addition salts of said para-aminophenols;
(c) at least one coupler chosen from meta-aminophenols, meta-phenylenediamines, and meta-diphenols, and acid addition salts of said couplers; and
(d) at least one enzyme of the laccase type;
provided that when said composition comprises at least one second oxidation base chosen from para-aminophenols, then the coupler is not chosen from 4-chlororesorcinol, meta-phenylenediamine, and their acid addition salts; and
provided that when said composition comprises at least one first oxidation base chosen from para-tolylene-diamines and at least one second oxidation base chosen from para-aminophenols, then the coupler is not chosen from 5-amino-2-methylphenol and resorcinol.

55. A method for dyeing keratinous fibers comprising the steps of:
(a) storing a first composition;
(b) storing a second composition separately from said first composition;
(c) mixing said first composition with said second composition to form a mixture; and
(d) applying said mixture to said keratinous fibers for a sufficient time to achieve a desired coloration;
wherein said first composition comprises at least one first oxidation base chosen from para-phenylenediamines and their acid addition salts, at least one second oxidation base chosen from para-aminophenols and their acid addition salts, and at least one coupler chosen from meta-aminophenols, meta-phenylenediamines, and meta-diphenols, and acid addition salts of said couplers, in a medium appropriate for dyeing keratinous fibers; provided that when said composition comprises at least one second oxidation base chosen from para-aminophenols, then the coupler is not chosen from 4-chlororesorcinol, meta-phenylenediamine, and their acid addition salts; and provided that when said composition comprises at least one first oxidation base chosen from para-tolylene-diamines and at least one second oxidation base chosen from para-aminophenols, then the coupler is not chosen from 5-amino-2-methylphenol and resorcinol; and
wherein said second composition comprises at least one enzyme of the laccase type, in a medium appropriate for dyeing keratinous fibers.

56. A multicompartment device or a dyeing kit, comprising:
a first compartment containing a first composition comprising at least one first oxidation base chosen from para-phenylenediamines and their acid addition salts, at least one second oxidation base chosen from para-aminophenols and their acid addition salts, and at least one coupler chosen from meta-aminophenols, meta-phenylenediamines, and meta-diphenols, and acid addition salts of said couplers, in a medium appropriate for dyeing keratinous fibers; provided that when said composition comprises at least one second oxidation base chosen from para-aminophenols, then the coupler is not chosen from 4-chlororesorcinol, meta-phenylenediamine, and their acid addition salts; and provided that when said composition comprises at least one first oxidation base chosen from para-tolylene-diamines and at least one second oxidation base chosen from para-aminophenols, then the coupler is not chosen from 5-amino-2-methylphenol and resorcinol; and
a second compartment containing a second composition comprising at least one enzyme of the laccase type, in a medium appropriate for dyeing keratinous fibers.
